(19) United States
(12) Patent Application Publication
(10) Pub. No.: US 2016/0038573 A1
(43) Pub. Date: Feb. 11, 2016

(54) SELECTIVE CELL-DEATH-INDUCING BINARY ENZYME SYSTEM

(71) Applicant: SIT-SOFT INTELLIGENT THERAPEUTICS GMBH & CO. KG, Dortmund (DE)

(72) Inventor: Sylvain TOUREL, Hattingen (DE)

(73) Assignee: SIT-Soft Intelligent Therapeutics GmbH & Co. KG, Dortmund (DE)

(21) Appl. No.: 14/761,632

(22) PCT Filed: Jan. 17, 2014
(86) PCT No.: PCT/EP2014/050966

§ 371 Date: Jul. 17, 2015

(30) Foreign Application Priority Data
Jan. 17, 2013 (EP) 13151753.4

(51) Int. Cl. A61K 38/48; C12N 9/64

(52) U.S. Cl. CPC A61K 38/482; A61K 38/4873; C12N 9/6467; C12Y 304/21079; C12Y 304/22044; A61K 48/00

(57) ABSTRACT

The invention relates to a combination preparation containing a selective cell death-inducing binary enzyme system for use in the therapy and/or treatment of cancer and tumors in humans and animals, a process, and its use.

The invention relates to a combination preparation containing a selective cell death-inducing binary enzyme system for use in the therapy and/or treatment of cancer and tumors in humans and animals, a process, and its use.

Cancer is a class of diseases that are characterized by uncontrolled cell growth and the dissemination of degenerate cells in the body and, in the case of metastasis, ultimately lead to the death of the patient. The treatment of tumors and cancer diseases depends strongly on the type of the tumor that appears and today usually involves the use of radiation therapy or chemotherapy, in addition to invasive surgery. Cancer diseases are triggered both by external factors (tobacco smoking, infectious organisms or viruses, mutagens, and ionizing radiation) and also by internal factors (genetic predisposition, hormones, immune system factors and spontaneous somatic mutations). Cancer can also be treated by immunotherapy, hormone therapy, and also by targeted therapy. The advantages of using chemotherapy to kill tumor cells are justified by its ability to interrupt cell division by exerting a destructive effect on the cellular DNA or RNA. As soon as the tumor cells can no longer divide, they die. The more quickly the cells divide, the higher the probability that they can be killed by the chemotherapeutic agent and [that] a tumor will shrink by the induction of cell death. Consequently, chemotherapy acts most efficiently on cells that divide quickly. However, chemotherapy is unable to distinguish between cancer/tumor cells and rapidly growing normal cells of the body, so that side effects such as hair loss, fatigue, pain, blood count changes, and nausea occur. Chemotherapy is divided into five large classes based on the mechanism of action: alkylating agents, plant alkaloids, antitumor antibiotics, and antimetabolites.

So-called targeted therapies exploit our knowledge of the differences of cancer cells from normal healthy cells. Targeted therapy is intended to eliminate cancer cells by exploiting specific features of these cancer cells so that there is no damage to normal, healthy cells. The active ingredients of such targeted therapies comprise especially monoclonal antibodies that specifically recognize and bind to the cancer cells, and angiogenesis inhibitors that specifically inhibit the growth of the blood vessels that supply the tumor. For the most part, targeted therapy uses small organic molecules that can penetrate the cancer cell membrane and block cellular metabolism, and especially to trigger apoptosis, killing the cells. A number of active ingredients have been described that target intracellular signal pathways to trigger such apoptosis. Other active ingredients recognize and bind to tumor-specific receptors on the cell surface.

However, these therapies place an extraordinary burden on the immune system, and in many cases can only be used to a limited extent. In addition, for the most part these forms of therapy require long pauses between the individual treatments for regeneration of the immune system. Therefore, in recent years especially gene therapy approaches or genetic vaccination have turned out to be promising for treatment, or in support of these classic measures.

Gene therapy and genetic vaccination are molecular medical procedures whose general use in the therapy and prevention of diseases have considerable impact on medical practice. Both procedures are based on the introduction of nucleic acids or peptides into the patient's cells or tissue, and on these cells or tissue then processing the information encoded by the introduced nucleic acids, i.e., on the expression of the desired polypeptides.

The usual approach of existing gene therapy and genetic vaccination procedures is to use DNA to introduce the required genetic information into the cell. In this connection, various procedures have been described to introduce DNA into cells, such as calcium phosphate transfection, Polybrene® transfection, protoplast fusion, electroporation, microinjection, and lipofection.

Another procedure that has been proposed, especially for genetic vaccination, is the use of DNA viruses as a DNA vehicle. Such viruses have the advantage that their infectious properties allow them to achieve a very high transfection rate.

Proteases are special proteins with peptidolytic and esterolytic properties that can irreversibly change and convert other substances and proteins (substrates) by catalysis. These proteases are divided into four different main classes on the basis of the functionally relevant molecular group of the catalytically active center: serine proteases, cysteine proteases, aspartate proteases, and metalloproteases. Serine proteases fall into two large families: the family of true serine proteases and the subtilisin family. The best-known representatives of the serine proteases include the digestive enzymes of the gastrointestinal tract (trypsin, chymotrypsin, and pancreatic elastase), the bactericidal and matrix-digesting enzymes of neutrophils (leukocyte elastase and cathepsin G), the kallikreins of the salivary glands, and the serine proteases of the coagulation system and immune defense system. Serine proteases in secretory granules of mast cells, lymphocytes, phagocytes, or natural killer cells and the serine proteases of the complement system play an important role in immune defense from viruses, parasites, bacteria, and tumor cells, and in autoimmune processes. Serine proteases have specificity for different substrates and can hydrolyze a peptide bond after aspartate groups (granzyme B, induction of DNA fragmentation in lysed target cells), arginine and lysine groups (trypsin, granzyme A, and granzyme K), methionine groups (granzyme M, met-ase), or after hydrophobic amino acids (elastase, proteinase 3, pancreatic elastase, chymotrypsin). A series of lymphocyte-specific serine proteases (called granzymes) are secreted during target cell lysis, and after absorption into the cytosol of the target cell are directly and indirectly involved in the process of target cell destruction by activated killer cells.

Apoptosis of a cell can be induced by various proapoptotic mechanisms and proteins. What these mechanisms and proteins have in common is that they activate a cascade of proteolytic cysteine proteases, called caspases, directed against cells. This cascade involves the initially activated caspases, such as, for example, caspase 8 and caspase 9, activating the effector cascade, such as, for example, caspases 3 and 6. These in turn cleave a series of cellular substrates, causing the apoptosis of the affected cell.

In the context of this invention, the term “programmed cell death” can be used as a synonym for “apoptosis”. As defined in this invention, an “induced cell death” is one in which an active substance triggers apoptosis or programmed cell death, preferably by means of a serine protease.

However, it is known that serine proteases can be used for tumor treatment.

There are a series of proteases that are only enzymatically active on substrate proteins that have a specific recognition sequence. The following table lists some examples. P1 designates the position of the amino acid after which the cleavage takes place, P4, P3, and P2 are the N-terminal positions before the restriction site P1. P1′ and P2′ are the C-terminal positions following Pl. This means that the proteases cleave the polypeptide chain between P1 and P1′.

TABLE 1

| | Restriction site | | | | | |
|---|---|---|---|---|---|---|
| Protease | P4 | P3 | P2 | P1 | P1′ | P2′ |
| Caspase 1 | F, W, Y or L | — | H, A or T | D | not P, E, D, | — |
| | | | | | Q, K or R | |
| Caspase 2 | D | V | A | D | not P, E, D, | — |
| | | | | | Q, K or R | |
| Caspase 3 | D | M | Q | D | not P, E, D, | — |
| | | | | | Q, K or R | |
| Caspase 4 | L | E | V | D | not P, E, D, | — |
| | | | | | Q, K or R | |
| Caspase 5 | L or W | E | H | D | — | — |
| Caspase 6 | V | E | H or I | D | not P, E, D, | — |
| | | | | | Q, K or R | |
| Caspase 7 | D | E | V | D | not P, E, D, | — |
| | | | | | Q, K or R | |
| Caspase 8 | I or L | E | T | D | not P, E, D, | — |
| | | | | | Q, K or R | |
| Caspase 9 | L | E | H | D | — | — |
| Caspase 10 | I | E | A | D | — | — |
| Clostripain | — | — | — | R | — | |
| (Clostridiopeptidase B) | | | | | | |
| Enterokinase | D or N | D or N | D or N | K | — | — |
| Factor Xa | A, F, G, I, L, | D or E | G | R | — | — |
| | T, V or M | | | | | |
| Granzyme B | I | E | P | D | — | — |
| Staphylococcus | — | — | not E | E | — | — |
| Peptidase I (V8 | | | | | | |
| Protease) | | | | | | |
| Thrombin | — | — | G | R | G | — |
| | A, F, G, I, L, | A, F, G, I, L, | P | R | not D, E | not D, E |
| | T, V or M | T, V, W or A | | | | |

Amino acids designated using one-letter code

An especially effective and specific serine protease (see Table 1) is granzyme B, as shown in SEQ ID no. 1.

Granzyme B (GzmB) is one of the main components of the granules of cytotoxic T cells (abbreviated as: CTL; obsolete term: T-killer cells). CTLs are able to transfer active GzmB to target cells, such as virus-infected cells or tumor cells, so that apoptosis is triggered. However, the recognition of target cells by CTL requires that the antigens be ingested and processed by dendritic cells (DC), which present them on major histocompatibility complex molecules and migrate to the local lymph nodes, so that antigen-specific T cells can be activated.

GzmB is a serine protease having the highly conservative N-terminal consensus sequence Ile-(Ile/Val)-Gly-Gly, which is essential for attaining the active conformation. For GzmB to change into its active form (referred to below as “active GzmB”), the N-terminal consensus sequence Ile-(Ile/Val)-Gly-Gly must be made available, as given in SEQ ID No. 2.

Starting from this prior art, the inventor's goal was to bring about the induced cell death of a cancer or tumor cell by means of an active ingredient.

Surprisingly, it is possible for tumor cells to die by means of a cell death-inducing binary enzyme system comprising a combination containing an inactive form of granzyme B comprising SEQ ID No. 2, or a nucleic acid encoding it, and tobacco etch virus protease (abbreviated as TEV) (SEQ ID no. 4 or SEQ ID no. 5), or a nucleic acid encoding it.

According to the invention, TEV recognizes the recognition site (recognition sequence) ENLYFQ (SEQ ID no. 6) or ENLYFQG (SEQ ID no. 7) in the inactive form, which is linked (ligated) with the N-terminus of SEQ ID no. 2. However, according to the invention, it is preferable for ENLYFQ (SEQ ID no. 6) to be ligated with the N-terminus of SEQ ID no. 2, which surprisingly gives improved cleavage.

Therefore, the goal is achieved in its full scope by the claims that have been drawn up.

As soon as the inactive form of granzyme B and TEV are introduced, either together or separately from one another, into a tumor cell and expressed (if applicable), TEV releases the active form of granzyme B comprising SEQ ID No. 2, inducing cell death through apoptosis or programmed cell death.

The inventive selection of the serine protease used in the invention and the means used, namely TEV, to unmask an inactive form of granzyme B into an active form, are especially advantageous. As soon as these two polypeptides are present in a tumor cell, the unmasking proceeds in a completely specific and efficient manner. Here it is especially advantageous that neither pre-granzyme B nor TEV occur in humans or mammals.

TEV is referred to in the document Kapust et al, The P1′ specificity of tobacco etch virus protease, Biochemical and Biophysical Research Communications, 294 (2002) 949-955.

Therefore, the invention relates to a drug or combination preparation comprising an inactive form of granzyme B comprising SEQ ID no. 2, or a nucleic acid encoding it, and TEV (e.g., SEQ ID no. 4 or SEQ ID no. 5), or a nucleic acid encoding it. TEV recognizes the recognition site (recognition sequence) ENLYFQ (SEQ ID no. 6) or ENLYFQG (SEQ ID no. 7) in the inactive form of granzyme B.

In a preferred embodiment of the invention, the inactive form of granzyme B is a pre-granzyme B (SEQ ID no. 3) or a nucleic acid encoding it (Zhinan Xia et al, Expression and Purification of Enzymatically Active Recombinant Granzyme B in a Baculovirus System, Biochemical and Biophysical Research Communications, 243 (1998) 384-389). An example of such an encoding nucleic acid is SEQ ID no. 8.

In another preferred embodiment of the invention, the inactive form of granzyme B is a fusion protein comprising SEQ ID no. 2 or a nucleic acid encoding it, wherein SEQ ID no. 2 is obtained or released through cleavage by TEV (e.g., SEQ ID no. 4 or SEQ ID no. 5) at ENLYFQ (SEQ ID no. 6) or ENLYFQG (SEQ ID no. 7).

Therefore, the invention relates to an inactive form of granzyme B, namely a fusion protein comprising SEQ ID no. 2 or a nucleic acid encoding it, wherein SEQ ID no. 2 is released through cleavage by TEV (e.g., SEQ ID no. 4 or SEQ ID no. 5) at the recognition sequence ENLYFQ (SEQ ID no. 6) or ENLYFQG (SEQ ID no. 7).

Therefore, the invention relates to an inactive form of granzyme B, namely a fusion protein comprising SEQ ID no. 2 and ENLYFQ (SEQ ID no. 6) or ENLYFQG (SEQ ID no. 7), or a nucleic acid encoding it, and possibly other sequences, wherein ENLYFQ (SEQ ID no. 6) or ENLYFQG (SEQ ID no. 7) is linked with the N-terminus of SEQ ID. no. 2. A corresponding embodiment is given in SEQ ID no. 3, namely of the type: FLAG-Tag-ENLYFQ-SEQ ID no. 2. Any other fusion proteins can be prepared in a corresponding manner (e.g., by means of an HIS-tag, and others), wherein the sample FLAG-tag can be replaced by any peptide, for example, 50 to 100 amino acids.

The person skilled in the art is able to produce and design suitable fusion proteins (Ausubel et al. (ed.), (1989). Preparation of Genomic DNA from Mammalian Tissue. In: Short Protocols in Molecular Biology: A Compendium of Methods from CURRENT PROTOCOLS IN MOLECULAR BIOLOGY. John Wiley & Sons).

The inventive combination preparations and drugs can have suitable excipients and additives added to them. Examples of suitable additives and/or excipients are, e.g., physiological saline solution, stabilizers, proteinase inhibitors, nuclease inhibitors, etc.

Therefore, the invention also relates to a combination preparation or drug as described above for application or use in the treatment and/or prophylaxis of cancer or tumor diseases in humans and animals, especially mammals.

In another preferred embodiment, the inventive combination preparations or drugs are administered by means of a gene therapy process.

Gene therapy processes can be obtained, e.g., by complexing the inventive nucleic acids with liposomes. Lipid mixtures suitable for this purpose are described by Feigner, P. L. et al. (1987) Proc. Natl. Acad. Sci, USA 84, 7413; Behr, J. P. et al. (1989) Proc. Natl. Acad. Sci. USA 86, 6982; Feigner, J. H. et al. (1994) J. Biol. Chem. 269, 2550, or Gao, X. & Huang, L. (1991) Biochim. Biophys. Acta 1189, 195. When the liposomes are produced, the DNA is ionically bound to the surface of the liposomes, and in such a ratio that a positive net charge remains, and the DNA is completely complexed by the liposomes. Sterically stabilized liposomes with a polyethylene glycol (PEG) shell exhibit clearly reduced ingestion through the mononuclear phagocyte system (MPS), and also have greatly prolonged blood circulation times, reduced aggregation of PEGylated vesicles, and improved stability of the liposomal formulations. Analogous to PEG, linear and hyperbranched polyglycerol (lPG and hbPG) show excellent biocompatibility, but allow further derivatives to be formed by the addition of functional groups. Novel lipids based on hyperbranched polyglycerol, linear-hyperbranched PEG-hbPG-block copolymers and statistical PEG-PG-copolymers were produced through combined anionic polymerizations of various epoxide monomers using lipophilic initiators such as cholesterol or 1,2-bis-n-alkyl glyceryl ethers. The novel amphiphilic structures were successfully introduced into liposomal membranes using 1,2-dioleoyl-sn-glycero-3-phosphocholine (DOPC) as a colipid.

Therefore, the invention also relates to a gene therapy process involving delivery into a target cell, preferably a tumor cell, by using a vehicle.

In another embodiment, this vehicle can be selected from the group of liposomes, nano- or microparticles, viruses, lipoplexes, etc. (Gene delivery by lipoplexes and polyplexes. Tros de Ilarduya C, Sun Y, Düzgüne [symbol] N. Eur J Pharm Sci. 2010 Jun. 14; 40(3):159-70. doi: 10.1016/j.ejps.2010.03.019. Epub 2010 Mar. 30; Efficient gene delivery by EGF-lipoplexes in vitro and in vivo, Buñuales M, Düzgüne [symbol] N, Zalba S, Garrido M J, de Ilarduya C T. Nanomedicine (Lond). 2011 January; 6(1):89-98. doi: 10.2217/nnm.10.100; Genetic nanomedicine: gene delivery by targeted lipoplexes, Düzgüne [symbol] N, de Ilarduya C T. Methods Enzymol. 2012;509:355-67. doi: 10.1016/B978-0-12-391858-1.00018-6).

In an especially preferred embodiment, the inventive vehicles have ligands on the surface that recognize tumor markers. Examples of such ligands are polyclonal or monoclonal antibodies or covalent binders (aptamers) that are able to bind to tumor markers.

Finally, such presenting tumor markers cannot be:

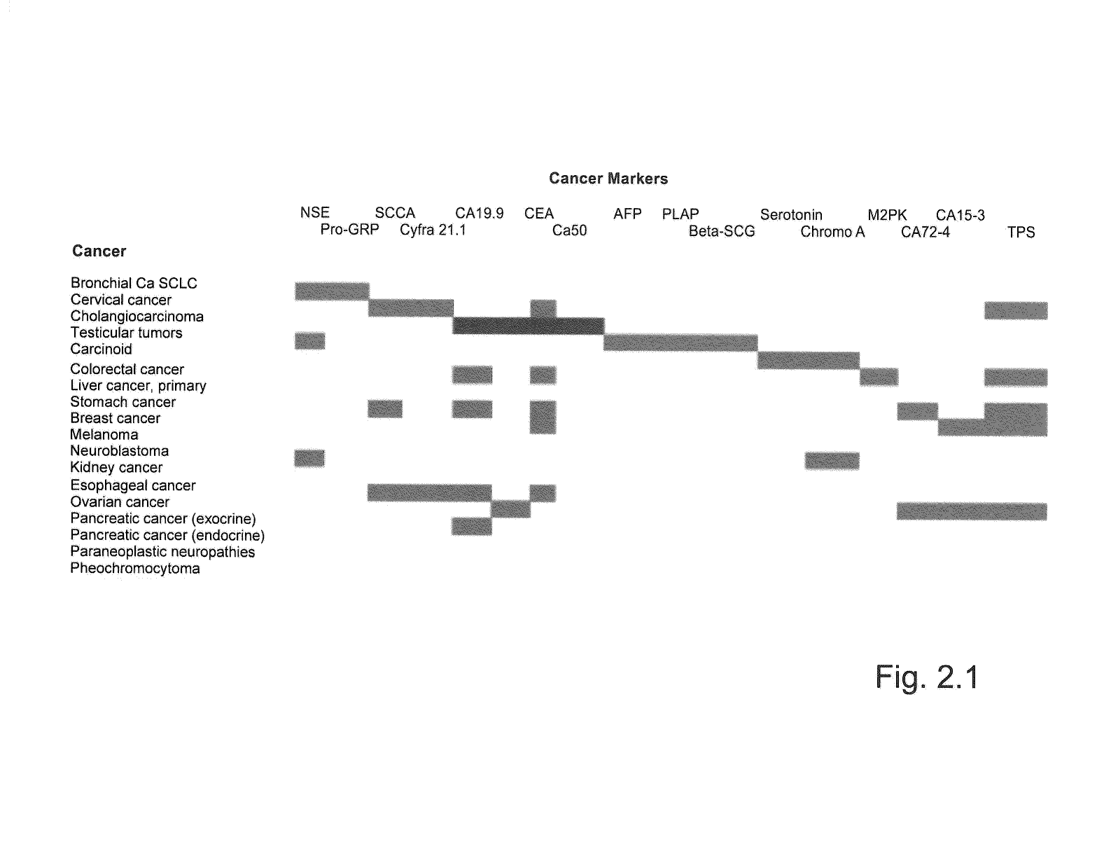

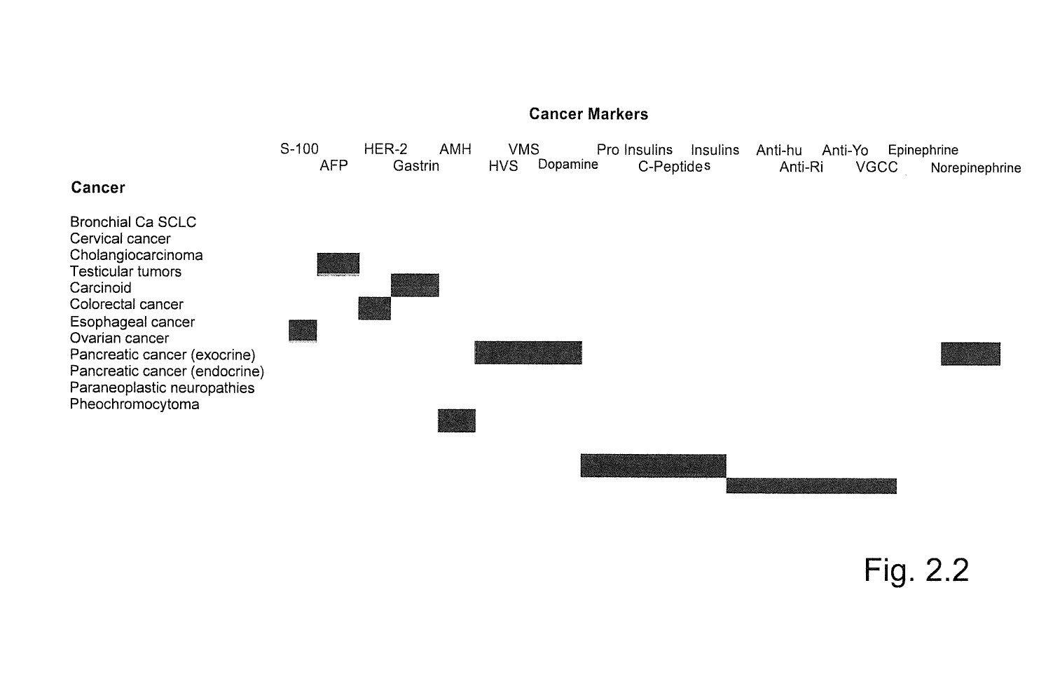

Carcinoembryonic antigen (CEA), alpha fetoprotein (AFP), carbohydrate antigen 19-9 (CA19-9), cancer antigen 72-4 (CA 72-4), cancer antigen 125, cancer antigen 15-3 (CA 15-3), neuron-specific enolase (NSE), squamous cell carcinoma antigen (SCC), cytokeratin fragment (CYFRA), human chorionic gonadotropin (HCG), prostate-specific antigen (PSA), human thyroglobulin (HTG), mucin-like cancer associated antigen (MCA), etc. FIG. 2 shows examples of tumor markers and the cancers for which they are suitable.

Therefore, the invention also relates to a process for introducing an inventive drug or combination preparation, wherein an inactive form of granzyme B comprising a nucleic acid encoding SEQ ID no. 2, and a nucleic acid encoding tobacco etch virus protease (e.g., SEQ ID no. 4 or SEQ ID no. 5), especially an inactive form of granzyme B comprising a nucleic acid encoding a fusion protein comprising SEQ ID no. 2 and ENLYFQ (SEQ ID no. 6) or ENLYFQG (SEQ ID no. 7) and a nucleic acid encoding tobacco etch virus protease,

i.) are introduced, together or separately from one another, in at least one vehicle,

ii.) into a tumor cell and expressed there,

iii.) producing an active form of granzyme B and inducing cell death in the tumor cell.

The process can be correspondingly adapted by other previously mentioned embodiments. The inventive drugs, combination preparations, and especially their vehicles can preferably be locally administered to humans and animals, e.g., subcutaneously administered. Of course the invention comprises all applications in tumor treatment.

As defined in this invention, the term “functional variant” is understood to mean polypeptides or nucleic acids that are functionally related with the inventive peptide. The term variants is also understood to mean allelic variants or polypeptides and nucleic acids that are derived from other organisms, cells, or tissues.

More broadly, it is also understood to mean polypeptides or nucleic acids that have a sequence homology, especially a sequence identity, of about 70%, preferably about 80%, especially preferably about 90%, most preferably about 95% with the designated SEQ ID.

This also includes polypeptide deletion in the range of about 1-50, preferably about 1-30, especially preferably about 1-15, most preferably about 1-6 amino acids. For example, the first amino acid can lack methionine, without substantially changing the function of the polypeptide.

In addition, this also includes fusion proteins that contain the above-described inventive polypeptides, the fusion proteins themselves already having the function of the respective SEQ ID or only being able to acquire the specific function after elimination of the fusion moiety. Above all, this includes fusion proteins whose component especially of non-human sequences is about 1-50, preferably about 1-30 amino acids. Examples of non-human peptide sequences are prokaryotic peptide sequences, e.g., from E. coli galactosidase or [those with] a so-called histidine tag, e.g., a Met-Ala-His6-Tag. An especially advantageous application for which fusion proteins with a so-called histidine tag are suitable is to purify the expressed protein through metal ion-containing columns, for example through a Ni²⁺-NTA column. Here “NTA” stands for the chelating agent nitrilotriacetic acid (Qiagen GmbH, Hilden).

Especially the mentioned parts of the polypeptide can also be synthesized using classical peptide synthesis (Merrifield method). They are especially suitable for obtaining antisera, which can be used to search through suitable gene expression libraries to achieve other functional variants of the inventive polypeptides.

In a preferred embodiment, the inventive nucleic acid previously mentioned in each case is a DNA, cDNA, or RNA, preferably a double-stranded DNA, however a PNA or something similar is also conceivable.

The inventive nucleic acids can also be introduced into the tumor cell by means of (expression) vectors, for example, by means of the vector pcDNA™ 3.1 (Invitrogen) with a constitutive CMV promoter, etc.

As defined in this invention, the terms tumor, cancer, cancer cells, and tumor cells should be read as synonyms, and comprise every benign or malignant tumor, especially a growth with a locally circumscribed increase in tissue volume, comprising every localized swelling due to edema, acute and chronic inflammation, aneurysmal enlargement (pulsating tumor) etc., and also inflammatory organ swelling (e.g., as in the case of a so-called splenic tumor) as well as a tissue neoplasm (growth, blastoma, neoplasia) in the form of a spontaneous, autonomous and irreversible excessive growth of the body's own tissue, disinhibited to different extents, which is, as a rule, connected with loss of specific cell and tissue functions of different severity (see Pschyrembel, (261st edition) 2007, de Gruyter, Berlin).

### EXAMPLES AND FIGURES

These examples serve exclusively to explain the invention, without limiting the it to these examples.

### EXAMPLE

### Example 1

Production of the pSTdna 1023 Plasmid

The tobacco etch virus protease sequence was ligated into the pcDNA™ 3.1 vector (Invitrogen company). In addition, the FLAG sequence (MDYKDDDDKGDYKDDDDKGGGT) was cloned into the n-terminal region twice.

Production of pSTdna 1024 (“GrzB WT”)

An additional plasmid based on the vector pcDNA™ 3.1 was cloned with the following sequence; this cloning was done twice FLAG sequence (MDYKDDDDKGDYKDDDDKGGGT) (SEQ ID no. 9) in combination with TEV recognition sequence (SEQ ID no. 6) and granzyme B AA21-240.

Production of the pSTdna 1025 Plasmid

The amino acid serine 203 in plasmid pSTdna 1024 was replaced by alanine 203. This mutation specifically inhibits granzyme B activity (“GrzB mut”).

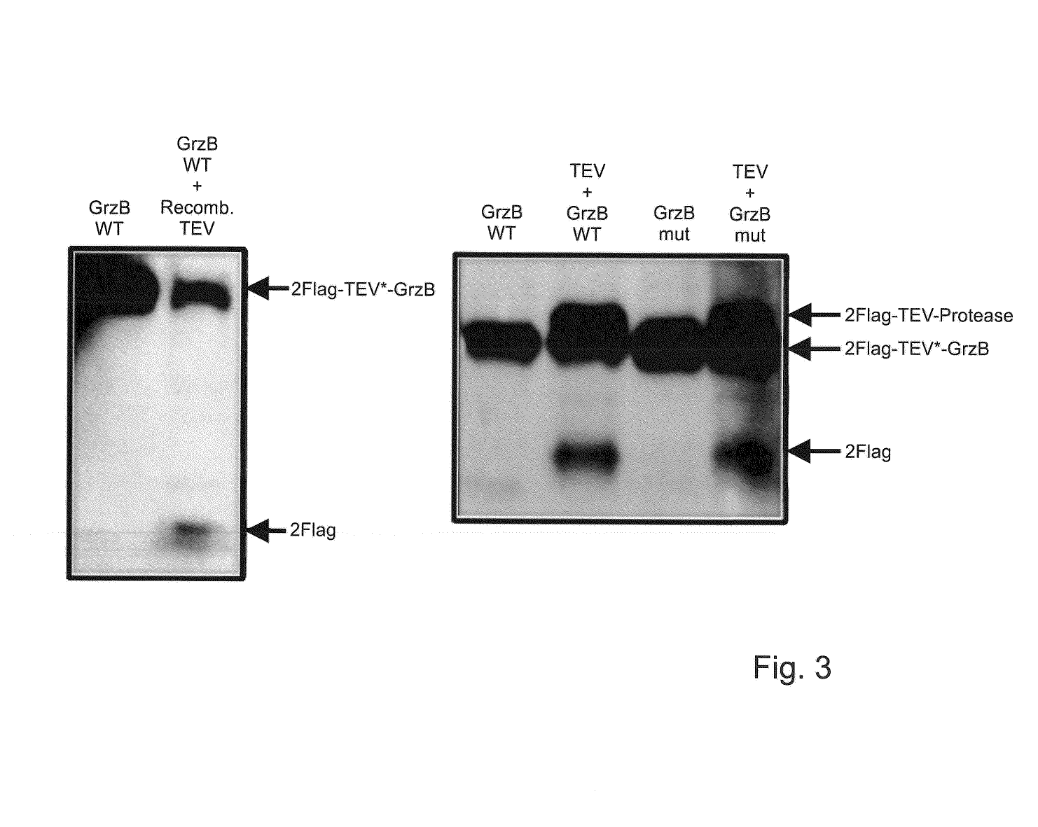

The results of cleavage with TEV are shown in FIG. 3. If the recognition site (SEQ ID no. 6) has a G instead of Q, no cleavage by TEV takes place (not shown).

### Example 2

Protocol

1. HeLa cells were cultivated in Dulbecco's modified Eagle's medium (DMEM) at 37° C. under 5% CO₂ to 75% confluence.

2. The medium was carefully aspirated and the cells were washed with phosphate buffered saline.

3. The cells were digested for 5 minutes using trypsin EDTA (200 mL/L), so that the cells were detached from the Petri dish. The digestion was stopped by adding DMEM.

4. The cells were centrifuged at 500 g for 5 minutes and resuspended with a 1 μg plasmid in a medium (Lonza) for electroporation. Then, the cells were electroporated with an Amaxa® Nucleofector™ II electroporator and resuspended in fresh DMEM.

5. The cells were incubated at 37° C. under 5% CO₂ for 4 h.

6. Solution A: 1 μg of plasmid was mixed with 100 μL of Opti-MEM® in a 1.5 mL tube.

7. Solution B: 8 μL of Lipofectamine® were mixed in a 1.5 mL tube and incubated at room temperature for 5 minutes.

8. Solutions A and B were pipetted together and carefully mixed. The incubation time is 30 minutes at room temperature.

9. The cell medium was carefully aspirated and the cells were washed twice with Opti-MEM®. The cells are incubated with 3 mL of Opti-MEM® at 37° C. under 5% CO₂.

10. The mixture of solutions A and B from step 8 was incubated for 30 minutes, and then AB was pipetted into the Opti-MEM® and carefully mixed.

11. The cells were incubated at 37° C. under 5% CO₂ for 5 hours.

12. After 5 h 5 mL of DMEM was added.

### DESCRIPTION OF THE FIGURES

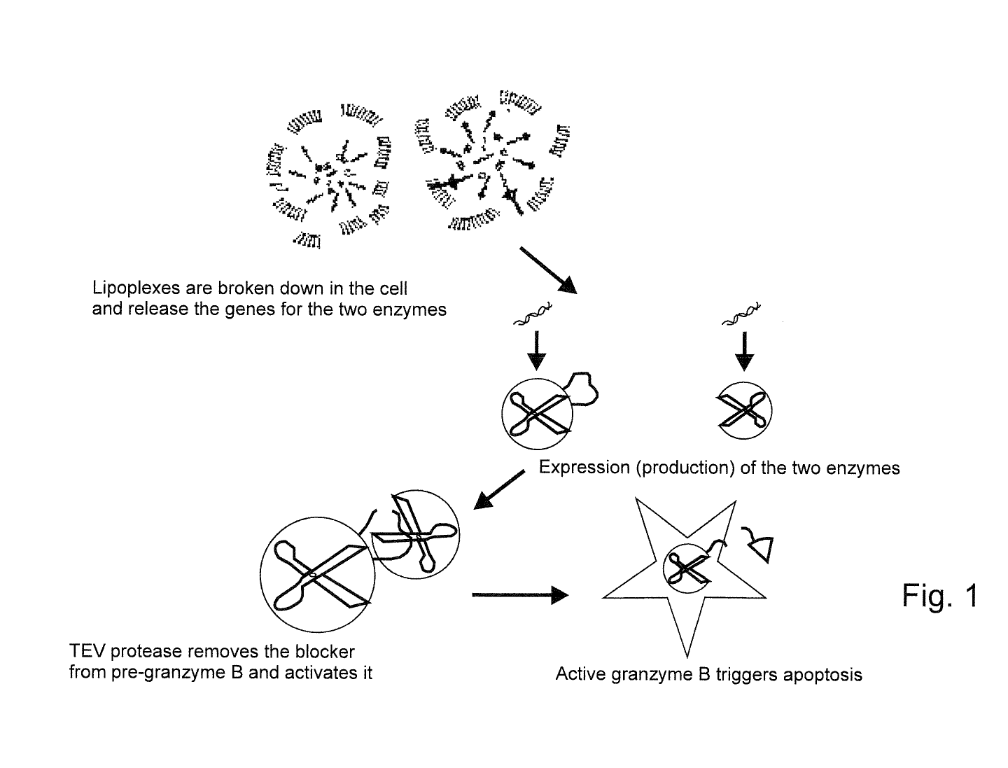

FIG. 1 describes the inventive therapeutic strategy to induce cell death of tumor cells.

FIG. 2 shows tumor markers for certain cancer diseases.

FIG. 3 shows TEV cleavage results from Example 1.

1-11. (canceled)

12. A drug for use in the treatment and/or prophylaxis of cancer or tumor diseases containing an inactive form of granzyme B comprising the sequence set forth in SEQ ID NO: 2 or a nucleic acid encoding it, or a functional variant of either, and tobacco etch virus protease comprising the sequence set forth in SEQ ID NO: 4 or SEQ ID NO: 5 or a nucleic acid encoding the tobacco etch virus protease or a functional variant of either, wherein the tobacco etch virus protease releases an active form of granzyme B comprising the sequence set forth in SEQ ID NO: 2.

13. The drug described in claim 12, wherein the inactive form of granzyme B is a pre-granzyme B comprising the sequence set forth in SEQ ID NO: 3 or a nucleic acid encoding the pre-granzyme B, or a functional variant of either.

14. The drug described in claim 12, wherein the inactive form of granzyme B is a fusion protein comprising the sequence set forth in SEQ ID NO: 2 or a nucleic acid encoding the sequence set forth in SEQ ID NO: 2, or a functional variant of either, wherein SEQ ID NO: 2 or a functional variant thereof is obtained by means of cleavage by tobacco etch virus protease comprising the sequence set forth in either SEQ ID NO: 4 or SEQ ID NO: 5 or a functional variant thereof.

15. A drug containing
an inactive form of granzyme B, wherein the inactive form of granzyme B is a fusion protein comprising the sequence set forth in SEQ ID NO: 2 or a nucleic acid encoding it, or a functional variant of either, wherein ENLYFQ (SEQ ID NO: 6) or ENLYFQG (SEQ ID NO: 7) is ligated with the N-terminus of SEQ ID NO: 2 and SEQ ID NO: 2 or a functional variant thereof is obtained or released by means of cleavage by tobacco etch virus protease at ENLYFQ (SEQ ID NO: 6) or ENLYFQG (SEQ ID NO: 7), and
tobacco etch virus protease or a nucleic acid encoding the tobacco etch virus protease or a functional variant of either.

16. A combination preparation containing a drug described in claim 12, and optionally excipients and additives.

17. The combination preparation described in claim 16 for use in the treatment and/or prophylaxis of cancer or tumor diseases in humans and animals.

18. A process comprising administering the combination preparation or drug of claim 12 by means of a gene therapy process.

19. A process comprising carrying out a gene therapy process by means of a vehicle comprising the combination preparation or drug of claim 12.

20. The process of claim 19, wherein the vehicle is selected from the group consisting of liposomes, nano- or microparticles, viruses, and lipoplexes.

21. The process of claim 19, wherein the vehicle further comprises ligands that recognize tumor markers.

22. A process for introducing a drug or combination preparation comprising
i) introducing an inactive form of granzyme B comprising a nucleic acid encoding a polypeptide having the sequence set forth in SEQ ID NO: 2, or a functional variant thereof, and a nucleic acid encoding tobacco etch virus protease or a functional variant thereof, together or separately from one another, in at least one vehicle,
ii.) introducing the vehicle into a tumor cell, wherein the inactive form of granzyme B is expressed in the tumor cell, and
iii.) producing an active form of granzyme B in the tumor cell and inducing cell death in the tumor cell.


SEQUENCE LISTING (9 sequences)

SEQ ID NO: 1 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 2 - protein, 229 aa, Homo sapiens
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGT

SEQ ID NO: 3 - protein, 260 aa, Artificial Sequence
  Homo sapiens
MDYKDDDDKGDYKDDDDKGGGTELGENLYFQIIGGHEAKPHSRPYMAYLMIWDQKSLKRCGGFLIRDDFVLTAAHCWGSSINVTLGAHNIKEQEPTQQFIPVKRAIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGT

SEQ ID NO: 4 - protein, 243 aa, tobacco etch virus
MGESLFKGPRDYNPISSTICHLTNESDGHTTSLYGIGFGPFIITNKHLFRRNNGTLLVQSLHGVFKVKDTTTLQQHLIDGRDMMIIRMPKDFPPFPQKLKFREPQREERICLVTTNFQAKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNELVYSQ

SEQ ID NO: 5 - protein, 262 aa, tobacco etch virus
MKHHHHHHPMSGLVPRGSAMGESLFKGPRDYNPISSTICHLTNESDGHTTSLYGIGFGPFIITNKHLFRRNNGTLLVQSLHGVFKVKDTTTLQQHLIDGRDMMIIRMPKDFPPFPQKLKFREPQREERICLVTTNFQAKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNELVYSQ

SEQ ID NO: 6 - protein, 6 aa, tobacco etch virus
ENLYFQ

SEQ ID NO: 7 - protein, 7 aa, tobacco etch virus
ENLYFQG

SEQ ID NO: 8 - DNA, 774 bp, Artificial Sequence
  Homo sapiens
atggnctacaaggacgacgatgacaagggtgactacaaggacgacgatgacaagggaggtggtaccgagctcggagaaaacctgtattttcagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctac

SEQ ID NO: 9 - protein, 22 aa, Artificial Sequence
  Sequence comprising two synthetically-derived FLAG epitope tags
MDYKDDDDKGDYKDDDDKGGGT